(19) United States
(12) Patent Application Publication
(10) Pub. No.: US 2009/0156648 A1
(43) Pub. Date: Jun. 18, 2009

(54) Preparations containing pyridoxine and alpha-hydroxyisocaproic acid (HICA)

(75) Inventors: Michele Molino, Mississauga (CA); Joseph MacDougall, Mississauga (CA)

Correspondence Address: TORYS LLP, 79 WELLINGTON STREET WEST, SUITE 3000, BOX 270, TD CENTRE, TORONTO, ON, M5K 1N2, CA

(73) Assignee: IOVATE T. & P. INC., Mississauga (CA)

(21) Appl. No.: 11/954,652

(22) Filed: Dec. 12, 2007

(51) Int. Cl. A61K 31/4412; C07D 211/90

(52) U.S. Cl. 514/348; 546/296

(57) ABSTRACT

The present invention relates to stable salts of pyridoxine and α-hydroxyisocaproic acid (HICA) endowed with enhanced nutritional and/or therapeutical efficacy in respect to their individual effects and to solid compositions containing such salts, particularly suited to oral administration. A method of preparation is also provided.

### RELATED APPLICATIONS

This application is related to the Applicant's co-pending U.S. patent application Ser. No. ______, entitled “Method for maintaining physiological pH levels during intensive physical exercise” filed on Dec. 12, 2007, the contents of which is hereby fully incorporated by reference in it's entirety.

### FIELD OF THE INVENTION

The present invention relates to a structure and method for producing stable salts of pyridoxine and α-hydroxyisocaproic acid (HICA). More specifically, formed salts of the present invention are particularly well suited for oral administration thereby providing enhanced nutritional and/or therapeutical efficacy in relation to the individual components alone.

### BACKGROUND OF THE INVENTION

Pyridoxine is often referred to as vitamin B6, however, it is actually only one of three components which constitute vitamin B6; the others being pyridoxal and pyridoxamine. The active form of pyridoxine in the body is pyridoxal 5-phosphate, which is a coenzyme for all transamination and some decarboxylation and deamination reactions. Furthermore, pyridoxal 5-phosphate is required as a coenzyme for all transamination reactions which occur in the body (Peterson D L, Martinez-Carrion M. The mechanism of transamination. Function of the histidyl residue at the active site of supernatant aspartate transaminase. J Biol Chem. 1970 Feb. 25; 245(4):806-13).

α-hydroxyisocaproic acid (HICA), is an end product of the metabolism of the branched chain amino acids, and is a nitrogen-free pre-cursor from which amino acids can be synthesized. Since branched chain amino acid analogs may be reaminated back to their correspondent amino acid (e.g. HICA can be converted to ketoisocaproic acid (KICA), which can subsequently be converted back to Leucine), they can act to provide the dietary requirements for BCAA without increasing level of ingested nitrogen (Boebek K P, Baker D H. Comparative utilization of the α-keto and D- and L-α-hydroxy analogs of Leucine, Isoleucine and Valine by chicks and rats. J Nutr. 1982 October; 112(10):1929-39). Transamination is the transfer of the amino group from an amino acid to an α-keto acid, e.g. α-ketoisocaproic acid can be converted to Leucine in this manner. As the product of transamination reactions depend on the availability of α-keto acids, providing exogenous HICA would make the formation of Leucine more favorable. Thus oral administration of analogues of branched-chain amino acids will increase the cellular content of the corresponding branched-chain amino acid, while substantially simultaneously reducing plasma and cellular ammonia.

There is now an extensive and ever growing body of literature, particularly patents, disclosing the formation of various salts having physiological functions in mammals.

UK Patent No. 1,248,324 (‘324’) discloses the formation of pyridoxine and α-ketoglutarate salts. α-Ketoglutarate is the deaminated form of glutamate, and is an intermediate in the citric acid cycle. Transamination of branch chain amino acids occurs primarily with α-ketoglutarate to form glutamate; however the reverse reaction of Glutamate to branch chain amino acids does not occur.

It would be desirable for the development of new salts of amino acid metabolites capable of being reaminated and transaminated to branch chain amino acids for use in the body of a mammal using the nitrogen found in the body of said mammal without the requirement of adding additional nitrogen to the system. It would therefore also be desirable to produce a compound having the aforementioned qualities while additionally providing required co-factors for transamination reactions to occur.

### SUMMARY OF THE INVENTION

In the present invention, a compound and methods for its production are disclosed. Specifically, the compound is a salt comprising a molecule of pyridoxine and a molecule of α-hydroxyisocaproic acid (HICA), and having a structure of Formula 1:

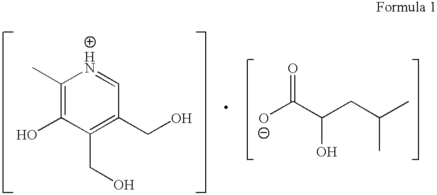

### DETAILED DESCRIPTION OF THE INVENTION

In the following description, for the purposes of explanations, numerous specific details are set forth in order to provide a thorough understanding of the present invention. It will be apparent, however, to one of ordinary skill in the art that the present invention may be practiced without these specific details.

The present invention is directed towards the structure and synthesis of salts of pyridoxine and α-hydroxyisocaproic acid (HICA).

The present invention provides for the production of a stable salt which may afford a synergistic combination of pyridoxine and HICA, free of physiologically unsafe additives to an individual upon administration to a mammal. Furthermore, the present invention is particularly well suited for use in tablets, capsules, powders, granules, powdered beverage mixes and other forms known in the art of dietary supplements.

As used herein, the term ‘pyridoxine α-hydroxyisocaproate’ is to be understood as the salt of pyridoxine with HICA reacted in an equimolar ratio.

Pyridoxine α-hydroxyisocaproate is a non-hygroscopic crystalline powder, which is stable in storage and can be processed without special precautions. Due to the non-hygroscopic nature of the pyridoxine α-hydroxyisocaproate it would be understood by one of skill in the art, that the salt is easy to process and is particularly suitable for processing with rapidly running machines, since it does not tend to stick together or become lumpy.

As used herein, ‘pyridoxine’ refers to the chemical 2-methyl-3-hydroxy-4,5-dihydroxymethylpyridine, (CAS Registry No. 65-23-6), also known as 3-hydroxy-4,5-bis(hydroxymethyl)-2-methylpyridine, 3-hydroxy-4,5-dimethyl-α-picoline, 5-hydroxy-6-methyl-3,4-pyridinedimethanol, or Vitamin B6. Additionally, as used herein, ‘pyridoxine’ also includes derivatives of pyridoxine such as esters, and amides, and salts, as well as other derivatives, including derivatives having substantially similar pharmacoproperties to pyridoxine upon metabolism to an active form.

As used herein, ‘α-hydroxyisocaproic acid’ refers to the chemical 2-hydroxy-4-methylvaleric acid, (CAS Registry No. 498-36-2), also known as HICA, or leucic acid. Additionally, as used herein, ‘α-hydroxyisocaproic acid’ also includes derivatives of α-hydroxyisocaproic acid such as esters, and amides, and salts, as well as other derivatives, including derivatives having substantially similar pharmacoproperties to α-hydroxyisocaproic acid upon metabolism to an active form.

As used herein, ‘lower alcohol’ refers to aliphatic alcohols having about 1 to about 4 carbon atoms as is known in the art, such as, without limitation, methanol, ethanol, propanol, and isopropanol. These lower alcohols may be used singly or in admixture containing two or more alcohols.

As used herein, ‘pharmaceutically acceptable excipients’ refers to substances added to produce quality tablets, chewable tablets, capsules, granulates or powders, but which do not provide nutritive value. A non-exhaustive list of examples of excipients includes monoglycerides, magnesium stearate, modified food starch, gelatin, microcrystalline cellulose, glycerin, stearic acid, silica, yellow beeswax, lecithin, hydroxypropylcellulose, croscarmellose sodium, and crospovidone.

According to the present invention, the compounds disclosed herein comprise an α-hydroxyisocaproic acid molecule combined with a pyridoxine molecule to form a salt having a structure according to Formula 1. The aforementioned compound being prepared according to the reaction as set forth for the purposes of the description in Scheme 1:

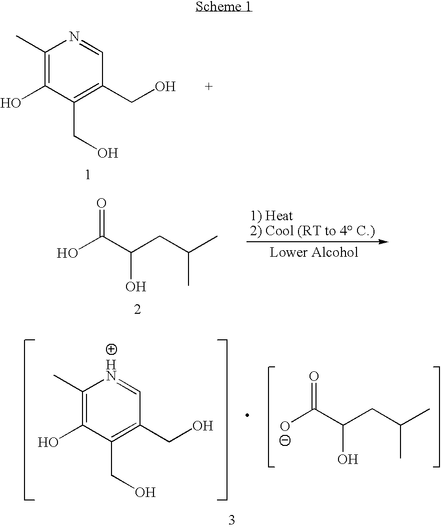

With reference to Scheme 1, in the first step of the reaction the pyridoxine (1) is dissolved in an excess of hot lower alcohol. The lower alcohol is considered to be hot when it is heated to a temperature below the boiling point of the corresponding lower alcohol.

In various embodiments of the present invention, the lower alcohol is selected from the group consisting of methanol, ethanol, propanol, and isopropanol. These lower alcohols may be used singly or in admixture containing two or more alcohols.

Concurrently, in the second step of the reaction the α-hydroxyisocaproic acid (2) is dissolved into an excess of hot lower alcohol. The lower alcohol is considered to be hot when it is heated to a temperature below the boiling point of the corresponding lower alcohol.

Both solutions above are then mixed together and heated to about the boiling point of the corresponding lower alcohol. If there are solids still present the solution is filtered at this temperature to remove unreacted starting materials. The solution is then allowed to cool to room temperature and then covered and placed in a refrigerator until crystallization occurs, preferably for between about 24 to about 48 hours. The resultant crystals are filtered under vacuum and washed with ice cold lower alcohol, yielding a crystalline powder, the pyridoxine α-hydroxyisocaproate (3).

In larger scale preparations of the present invention diethyl ether can be added until the cloud point, as would be known to one of skill in the art, is reached after the mixture is cooled to room temperature. This will facilitate greater precipitation of the product thus yielding more of the pyridoxine α-hydroxyisocaproate (3), which would be desired in industrial settings.

Pyridoxine α-hydroxyisocaproate is used advantageously alone or with additional active ingredients, such as, trace elements, vitamins, mineral substances, or other amino acids as well as, optionally, excipients usually used for the preparation of the respective forms of administration. The forms of administration include, particularly, all varieties of tablets, both those that are swallowed without being chewed, and tablets to be chewed or dissolved in the mouth of an individual, as well as those that are dissolved in a liquid before being ingested by an individual. The tablet forms include uncoated tablets, one-layer or multilayer or encased forms or effervescent tablets. Further preferred forms of administration are capsules of hard and soft gelatin, the latter having particularly suitable to include a liquid core. Additionally, pyridoxine α-hydroxyisocaproate can be used advantageously for the preparation of solutions and suspensions and as a powder, either effervescent or granulated.

While not wishing to be bound by theory, it is understood by the inventors that pyridoxine α-hydroxyisocaproate and its derivatives corresponding to Formula 1 above, are useful compounds, since they combine within a single molecule both the pyridoxine and the α-hydroxyisocaproate, thus resulting in the increase of the useful activities of these two compounds. Particularly, it is herein understood by the inventors that pyridoxine α-hydroxyisocaproate will have enhanced pH stability in water within a substantially broad range of concentrations.

The examples given below explain the execution of the invention with respect to the production of pyridoxine α-hydroxyisocaproate. Provided below is the a basic method of producing pyridoxine α-hydroxyisocaproate, however those of skill in the art will appreciate certain changes may be made in the process of “scaling-up” the reaction to manufacture larger batches of pyridoxine α-hydroxyisocaproate which may be required for commercial uses and supply requirements. Other methods of synthesis may also be apparent to those of skill in the art.

### EXAMPLES

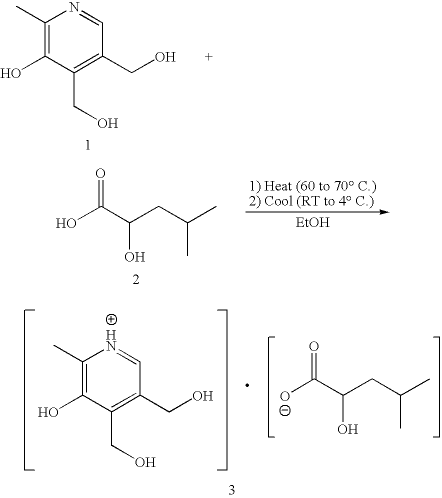

### Example 1

### Laboratory Scale

132.16 g (1 mol) of α-hydroxyisocaproic acid (2) is dissolved into 200 mL of hot ethanol, solution 1. Concurrently, 169.18 g (1 mol) of pyridoxine (1) is dissolved in 300 mL of hot ethanol, solution 2. Solution 2 is added to solution 1 with stirring and the resultant solution is heated to the boiling point. If there are solids still present the solution is filtered at this temperature to remove unreacted starting materials. The solution is then allowed to cool to room temperature and then covered and placed in a refrigerator until crystallization occurs; about 24 hours. The resultant crystals are filtered under vacuum and washed with ice cold ethanol, yielding a crystalline powder, the pyridoxine α-hydroxyisocaproate (3).

### Example 2

### Industrial Scale

781.179 kg (5910.86 mol) of α-hydroxyisocaproic acid (2) is dissolved into 800 L of hot ethanol, solution 3. Concurrently, 1000 kg (5910.86 mol) of pyridoxine (1) is dissolved in 1000 L of hot ethanol, solution 4. Solution 3 is added to solution 4 with stirring and the resultant solution is heated to the boiling point. If there are solids still present the solution is filtered at this temperature to remove unreacted starting materials. The solution is then allowed to cool to room temperature, diethyl ether is added until the cloud point is reached, and the mixture is then cooled for 48 hours to induce crystallization. The resultant crystals are then vacuum-filtered and washed with ice cold ethanol, yielding a crystalline powder, the pyridoxine α-hydroxyisocaproate (3).

Extensions and Alternatives

In the foregoing specification, the invention has been described with specific embodiments thereof; however, it will be evident that various modifications and changes may be made thereto without departing from the broader spirit and scope of the invention.

What is claimed:

1. A salt of pyridoxine and α-hydroxyisocaproic acid, having the Formula:

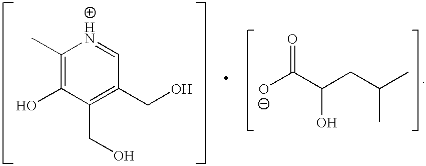

2. A composition comprising the compound of claim 1, further comprising pharmaceutically acceptable excipients.

3. The composition of claim of claim 2 wherein the pharmaceutically acceptable excipients are selected from the group consisting of monoglycerides, magnesium stearate, modified food starch, gelatin, microcrystalline cellulose, glycerin, stearic acid, silica, yellow beeswax, lecithin, hydroxypropylcellulose, croscarmellose sodium, and crospovidone.

4. The salt of pyridoxine and α-hydroxyisocaproic acid of claim 1 wherein said salt of pyridoxine and α-hydroxyisocaproic acid is provided in a dosage form selected from the group consisting of ingestible tablets, chewable tablets, capsules, granulates or powders.

5. The composition of claim 2 wherein the composition is in a dosage form selected from the group consisting of ingestible tablets, chewable tablets, capsules, granulates or powders.

6. The salt of pyridoxine and α-hydroxyisocaproic acid of claim 1 wherein said salt of pyridoxine and α-hydroxyisocaproic acid is administered to a mammal.

7. The composition of claim 4 wherein the composition is administered to a mammal.

8. The salt of pyridoxine and α-hydroxyisocaproic acid of claim 6 wherein said salt of pyridoxine and α-hydroxyisocaproic acid is orally administered to a said mammal.

9. The composition of claim 7 wherein the composition is orally administered to a said mammal.

10. A method for producing a pyridoxine α-hydroxyisocaproate salt comprising at least the steps of:
a) dissolving α-hydroxyisocaproic acid in hot lower alcohol;
b) dissolving pyridoxine in hot lower alcohol;
c) mixing the resultant solutions of a) and b);
d) cooling the resultant mixture; and
e) isolating the resulting pyridoxine α-hydroxyisocaproate salt.

11. The method of claim 10 wherein the lower alcohol is selected from the group consisting of methanol, ethanol, propanol, butanol, and isopropanol.

12. The method of claim 10 wherein the α-hydroxyisocaproic acid and the pyridoxine are present in an equimolar ratio.

13. The method of claim 10 wherein the resultant mixture is cooled until crystallization occurs.

14. The method of claim 13 wherein crystallization occurs between about 24 to about 48 hours following the commencement of the cooling.

15. The method of claim 10 wherein the pyridoxine α-hydroxyisocaproate salt is isolated by vacuum filtration followed by washing of the filtrate with cold lower alcohol.

16. The pyridoxine α-hydroxyisocaproate salt of claim 10 having the molecular structure of: [chemical structure]
